(19) United States
(12) Patent Application Publication
(10) Pub. No.: US 2021/0158722 A1
(43) Pub. Date: May 27, 2021

(54) SYSTEMS AND METHODS FOR ENCOURAGING PATIENT BEHAVIOR

(71) Applicant: ELYSIOT Inc., Milpitas, CA (US)

(72) Inventors: Bhavik VYAS, Milpitas, CA (US); Namasivayam THANGAVELU, San Jose, CA (US)

(21) Appl. No.: 17/112,388

(22) Filed: Dec. 4, 2020

Related U.S. Application Data
(63) Continuation of application No. PCT/US2019/035827, filed on Jun. 6, 2019.
(60) Provisional application No. 62/681,556, filed on Jun. 6, 2018.

(51) Int. Cl. G09B 19/00; A61J 7/04; A61B 5/00

(52) U.S. Cl. CPC G09B 19/00; A61J 7/0436; A61B 5/4833; A61B 5/7405; A61J 2200/30; A61B 5/486; A61J 2200/70; A61J 2205/70; A61B 5/742

(57) ABSTRACT

Various systems and methods for encouraging patient behavior utilize one or more sensors for gathering medical-related information about a patient and processing the medical-related information to provide feedback to the patient, such as notification reminders for dosing, testing, and/or other patient behaviors.

### CROSS-REFERENCE TO RELATED APPLICATIONS

This application claims priority to U.S. Provisional Application Ser. No. 62/681,556, filed on Jun. 6, 2018, the content of which is hereby incorporated by reference in its entirety.

### FIELD

This invention relates generally to the field of medical care, and more specifically to encouraging patient behavior for purposes of medical care.

### BACKGROUND

Successful medical treatment often requires patients to engage in and maintain certain behaviors, such as taking medication as prescribed by a medical professional, following a certain exercise regimen, or maintaining a prescribed diet. A patient's failure to engage in certain behaviors not only endangers the patient's health, but also results in increased healthcare costs and inefficiencies in the healthcare system. For example, it is estimated that patients' lack of compliance in taking medication as prescribed (e.g., by delaying or skipping doses) contributes to nearly 125,000 deaths and up to 10% of hospitalizations in the U.S. alone each year, and ultimately costing the U.S. healthcare system between $100-$289 billion per year. Thus, it is desirable to have improved systems and methods for encouraging patient behavior in order to improve health outcomes and mitigate strain on the healthcare system.

### SUMMARY

Generally, systems and methods for encouraging patient behavior may involve one or more sensors for gathering medical-related information about a patient and processing the medical-related information to provide feedback to the patient, such as notification reminders. In some variations, the medical-related information may be further processed within a cryptocurrency blockchain environment that facilitates the secure transfer of tokens that may be exchangeable for patient benefits or other benefits.

In some variations, a medication monitor includes an enclosure comprising a cavity configured to receive a medication container. A lid may have at least a portion that is removably coupleable to the enclosure such that, when the lid is coupled to the enclosure, the medication monitor substantially surrounds the medication container. An adjustable holder may be configured to position at least a portion of the medication container within the cavity of the enclosure. A sensor may be configured to generate medication data. A transmitter may be configured to transmit at least the medication data to one or more computing devices.

In some variations, the medication container may comprise a set of dimensions within a predetermined range of dimensions. In some variations, the holder may be configured to adjustably position at least the portion of the medication container having a set of dimensions within a predetermined range of dimensions. The medication data may indicate a quantity of medication.

In some variations, a sidewall of the enclosure may comprise the sensor. In some variations, the sensor may comprise a capacitance sensor. In some variations, the sensor may comprise an LC resonator. In some variations, a sensor may be configured to generate lid data indicating opening or closing of the lid relative to the enclosure. In some variations, the holder may be coupled to the enclosure. In some variations, the holder may be coupled to the lid.

In some variations, the holder may be configured to fix the position of the medication container relative to the enclosure. In some variations, the holder may comprise a compressible member configured to transition between an expanded configuration and a compressed configuration. In some variations, the compressible member may be configured to transition from the expanded configuration to the compressed configuration in response to placement of the medication container into the cavity of the enclosure.

In some variations, the compressible member may be configured to surround at least a portion of the medication container in the compressed configuration. In some variations, the holder may comprise an expandable member configured to transition between a deflated configuration and an expanded configuration. In some variations, the expandable member may be configured to transition from the deflated configuration to the expanded configuration to position at least the portion of the medication container within the cavity of the enclosure. In some variations, the expandable member may be configured to receive a fluid comprising one or more of liquid and gas.

In some variations, the enclosure may comprise a set of steps increasing in height toward a perimeter of the enclosure. In some variations, the enclosure may define a longitudinal axis and the holder may be configured to position the medication container about the longitudinal axis of the enclosure. In some variations, the holder may position the medication container asymmetrically within the cavity of the enclosure. In some variations, the holder may comprise a set of one or more springs. In some variations, the holder may comprise a flexible member. In some variations, a sidewall of the enclosure may comprise a transparent portion.

In some variations, an output device may be configured to provide feedback to a patient. In some variations, the output device may comprise one or more of a display and an audio device. In some variations, the feedback may comprise one or more of a reminder and warning based on a predetermined dosage schedule. In some variations, the feedback may comprise one or more of a medication data, location, and health care provider communication data.

In some variations, a system for encouraging patient behavior includes at least one sensor removably coupleable to a mediation container and configured to generate a sensor signal indicating opening or closing of the medication container when the sensor is coupled to the medication container, an output device configured to provide feedback to a patient in response to the sensor signal, and a transceiver configured to transmit container data over a network to one or more computing devices. The container data may be based at least in part on the sensor signal. Examples of suitable kinds of sensors include an accelerometer and a gyroscope, which may be configured to detect motion of a portion of a container (e.g., lid) to indicate opening or closing of the container.

The output device of the system, which may also be coupleable to the medication container, can include any suitable device that may communicate with the patient, such as a display or an audio device. The output device may be configured to provide any suitable sensory feedback (e.g., visual, auditory, tactile, etc.). For example, the output device may provide visual feedback such as a visual graphic or message on a display. As another example, the output device may provide auditory feedback such as an auditory message. In some variations, the auditory message may be subliminal and played at a frequency for being heard subconsciously, such as at about 10 Hz or about 5.5 Hz.

A method for encouraging patient behavior may include receiving a sensor signal indicating opening or closing of a medication container, where the sensor signal is generated by a sensor removably coupleable to the medication container, generating feedback to the patient based on the sensor signal, and providing the feedback to the patient at a mobile device associated with the patient. The feedback may include, for example, a displayed graphic or message and/or auditory information. In some variations, auditory feedback may be output at a frequency of about 10 Hz or about 5.5 Hz. Generally, the feedback may be generated at least in part by comparing information derived from the sensor signal to a predetermined health regimen. Furthermore, in some variations the method may include providing a reminder to the patient at the mobile device to engage in an action associated with a predetermined health regimen.

A method for encouraging patient behavior may include receiving medication data generated by a medication monitor of a patient using a computing device comprising a processor and memory. A set of one or more predetermined contacts may be notified based at least on the medication data using the computing device. Feedback to the patient may be output using the medication monitor based at least in response to the medication data.

In some variations, the feedback may comprise at least one prompt to modify the patient behavior. In some variations, the prompt may comprise encouragement to comply with a dosage schedule. In some variations, one or more predetermined contacts may comprise one or more of a health care professional, a patient's partner, family member, and support group. In some variations, a communication channel may be established between the computing device and a health care professional device in response to the medication data.

A method for using a blockchain distributed network for encouraging patient behavior may include receiving a patient behavior record over the blockchain distributed network, where the patient behavior record includes data indicating patient behavior related to a health regimen. The method may also include accessing a distributed ledger stored in a memory device, wherein the distributed ledger comprises a record of at least one smart contract associated with the patient behavior record, and determining whether the patient behavior record satisfies at least one condition of the smart contract. In some variations, the method may further include instructing the transfer of one or more tokens to a patient account associated with the patient, if the at least one condition of the smart contract is satisfied, and updating the distributed ledger based on the transfer of one or more tokens, through communication over the blockchain distributed network. The tokens may, for example, be associated with a cryptocurrency.

Generally, a system for using a blockchain distributed network for encouraging patient behavior may include a memory device and a processing device operatively coupled to the memory device, where the processing device may be configured to execute computer-readable code to perform various processes at least in part over a blockchain distributed network. For example, the processing device may be configured to receive a patient behavior record over the blockchain distributed network, where the patient behavior record includes data indicating patient behavior related to a health regimen. The processing device may further be configured to access a distributed ledger stored in a memory device, where the distributed ledger includes a record of at least one smart contract associated with the patient behavior record, and determine whether the patient behavior record satisfies at least one condition of the smart contract. In some variations, the processing device may further be configured to instruct the transfer of one or more tokens to a patient account associated with the patient, if the at least one condition of the smart contract is satisfied, and update the distributed ledger based on the transfer of one or more tokens, through communication over the blockchain distributed network. The tokens may, for example, be associated with a cryptocurrency.

The patient behavior record may include various suitable information from one or more health improvement devices (e.g., a device that includes one or more sensors for gathering medical-related information), such as information indicating patient compliance with the health regimen, including but not limited to information indicating intake of medication in accordance with a treatment plan. Suitable health improvement devices for providing such information may include, for example, a pedometer, a glucose monitoring device, a blood pressure monitor, a device with one or more sensors coupleable to a medication container, etc.

In some variations, the smart contract associated with the patient behavior record may define a token transaction in exchange for specified patient behavior. For example, a certain number of tokens may be exchanged for a sufficient level of patient compliance with the health regimen (e.g., consistently taking medication). In some variations, the transaction may involve the transfer of one or more tokens from a health improvement device account associated with a health improvement device to, for example, a patient account associated with the patient. Multiple health improvement device accounts may be connected to the patient account to transfer tokens in exchange for various kinds of patient behavior. Furthermore, one or more health improvement device accounts may be replenished by another token account, such as an insurer account associated with a medical insurance company.

### BRIEF DESCRIPTION OF THE DRAWINGS

FIG. 1 is a block diagram of an exemplary variation of a system for encouraging patient behavior.

FIG. 2 is a block diagram of an exemplary variation of a health improvement device.

FIGS. 3A and 3B are illustrative schematics of exemplary variations of a health improvement device coupled to a medication container.

FIG. 4 is a flowchart of an exemplary variation of a method for encouraging patient behavior.

FIGS. 5A-5F are exemplary variations of GUIs operable on a personal computing device for encouraging patient behavior.

FIG. 6 is a block diagram of an exemplary variation of a cryptocurrency blockchain environment.

FIG. 7A is a block diagram of an exemplary variation of a distributed blockchain network.

FIG. 7B is a schematic illustration of an exemplary variation of a blockchain network system.

FIG. 8 is flowchart of an exemplary variation of a method for encouraging patient behavior using a cryptocurrency blockchain environment.

FIG. 9 is a block diagram of an exemplary variation of an arrangement of token accounts including accounts for a patient and health improvement devices.

FIG. 10 is a block diagram of an exemplary variation of a medication monitor.

FIG. 11 is an exploded schematic diagram of an exemplary variation of a medication monitor and a medication container.

FIGS. 12A and 12B are schematic illustrations of exemplary variations of a holder in a medication monitor.

FIGS. 13A and 13B are schematic illustrations of additional exemplary variations of a holder in a medication monitor.

FIGS. 14A-14C are schematic illustrations of additional exemplary variations of a holder in a medication monitor.

FIG. 15A is a plot illustrating an exemplary relationship between capacitance and number of pills. FIG. 15B is a plot illustrating an exemplary relationship between capacitance and volume of medication.

FIG. 16 is flowchart of an exemplary variation of a method for encouraging patient behavior.

### DETAILED DESCRIPTION

Non-limiting examples of various aspects and variations of the invention are described herein and illustrated in the accompanying drawings.

Described herein are various systems and methods for encouraging patient behavior. For example, health improvement devices may interact with a user interface on a personal computing device configured to monitor, remind, and/or otherwise motivate patients to comply with a health regimen. For example, the health improvement devices may monitor medication and/or user interaction with the health improvement device. In some variations, the health improvement devices may be adapted to receive a medication container (e.g., prescription pill bottle), such as those having any standard or predetermined dimension. Furthermore, one or more predetermined contacts (e.g., health care professional, patient, caregiver, partner, etc.) may interact with a user interface on the personal computing device to encourage patient behavior. As another example, patient behavior may be encouraged in the context of a cryptocurrency system, such as by rewarding patients with tokens that may be redeemed for a benefit when patients engage in behavior in accordance with a health regimen. Thus, such systems and methods help emphasize healthy habits, adherence, and other behaviors, thereby increasing the likelihood that patients' health will be improved.

### Health Improvement Devices and User Interfaces

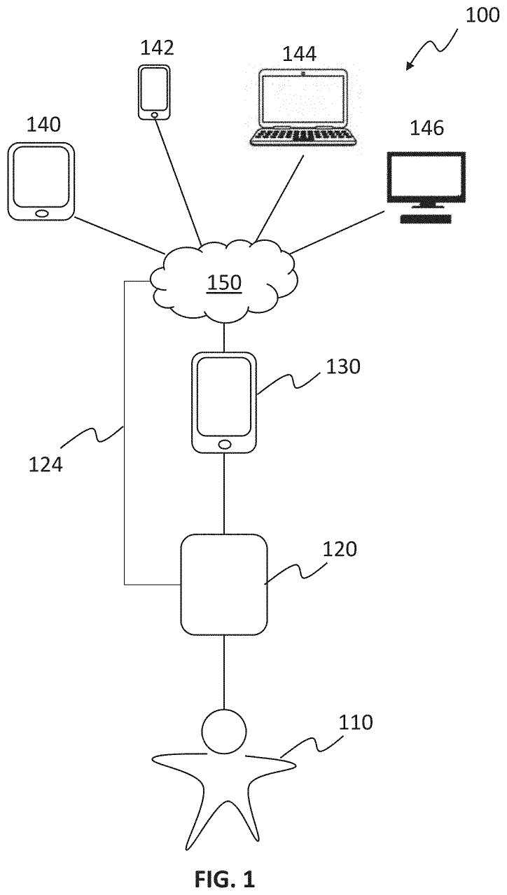

Generally, as shown in FIG. 1, a system 100 for encouraging patient behavior may include a health improvement device 120 that interfaces with patient 110 and may be configured to communicate with a personal computing device 130, which may have a user interface, computer application, or other program for engaging with a patient 110 as further described below. The health improvement device 120 may be configured to gather medical-related data (e.g., through one or more sensors) and communicate this data to the personal computing device 130 in any suitable wired or wireless (e.g., with Bluetooth or other suitable communication protocol). The personal computing device 130 may communicate in a wireless or wired manner with a network 150 (e.g., cloud-based network or other suitable network of computing devices), such that data received from the health improvement device 120 may be analyzed remotely (non-locally) from the personal computing device 130. Alternatively, data from the health improvement device 120 may be analyzed locally by one or more processors on the personal computing device 130. Additionally or alternatively, the health improvement device 120 may be configured to directly communicate with the network 150 through a direct communication link 124. For example, the health improvement device 120 may be connectable to the Internet, such as through a wireless or wired connection. The medical-related data (and/or information derived from the medical-related data) received by the network may be stored, for example, on one or more servers, and/or on one or more distributed systems such as across a blockchain distributed network similar to that described below.

In response to the received medical-related data, one or more computing devices may transmit feedback to the health improvement device 120 or computing device associated with the patient. For example, a patient may receive audio feedback on a patient's smartphone encouraging compliance with a predetermined dosage schedule. In some variations, the feedback may include audio and/or video generated by a predetermined contact of the patient such as a health care professional, caregiver, and partner. In some of these variations, feedback from a person having a personal and/or professional relationship with the patient may improve patient compliance with a health regimen.

In some variations, medical-related data about the patient (and/or information derived from the medical-related data) may be accessible by one or more third party computing devices. For example, as shown in FIG. 1, such data or information may be accessible by a third party computer device (e.g., tablet 140, mobile phone 142, laptop computer 144, desktop computer 146, etc.) that is in communication with the network 150. It should also be understood that any other computing device operated by the patient may similarly access the information over the network 150. The information (and use thereof) that may be accessible to other computing devices is further described below.

### Health Improvement Devices

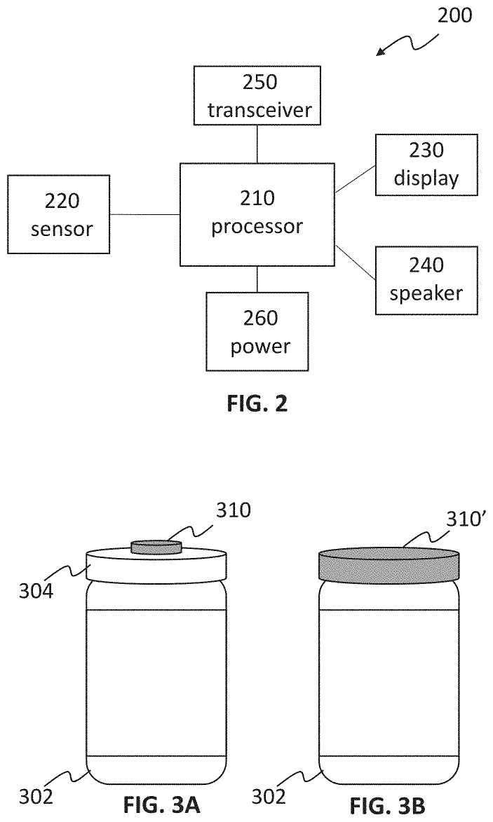

As shown in the general schematic of FIG. 2, in some variations, a health improvement device 200 may generally include at least one sensor 220 configured to generate a sensor signal indicating medical-related data associated with a patient, and at least one output device (e.g., display 230 and/or speaker 240) configured to provide feedback to a patient in response to the sensor signal. The health improvement device 200 may further include at least one transceiver 250 (or separate receiver and transmitter) for communicating the medical-related data to another computing device, such as a personal computing device (e.g., mobile phone or tablet, etc.). A processor 210 may be configured to receive a sensor signal from the sensor 220 and coordinate operation of other components such as the output devices and/or transceiver 250. Furthermore, the health improvement device 200 may include one or more power sources 260, such as a battery, which may be used to provide electrical power to the processor 210, sensor 220, and any other suitable electrical components.

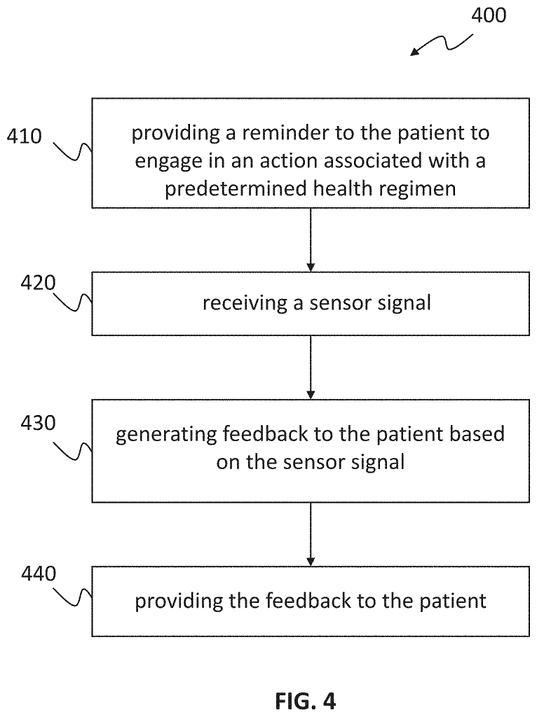

Health improvement devices may be used to gather various kinds of medical-related data about the patient. In some variations, the health improvement device may be configured to couple to a medication container. For example, FIG. 3A is a schematic illustration of one example of a health improvement device 310 that may detect indications of when a patient is taking medication, and may be used to encourage patient compliance with medication. Similar to that shown in FIG. 2, the health improvement device 310 may include at least one sensor, at least one output device (e.g., display and/or speaker), a transceiver, and a processor for coordinating the other components. The health improvement device 310 may include one or more sensors that are coupled to a portion of a medication container, such as a container lid 304. For example, as shown in FIG. 3A, the device 310 may be coupled to the top of the container lid 304, though it should be understood that alternatively at least a portion of the device 310 may be coupled to a side and/or underside of the container lid 304. The one or more sensors may be configured to detect when the medication container is opened or closed, such as by detecting when the container lid 304 is separated from the container body 302. In some variations, the one or more sensors health improvement device 310 may be configured to detect a certain motion associated with opening of the container (e.g., axial rotation of a threaded lid, lateral translation of a sliding lid, rotation of a flip-top lid around a lateral axis, etc.). In these variations, the health improvement device 310 may be used with a variety of containers having the same modality. For example, the health improvement device 310 may be configured to couple to any suitable container having the appropriate detectable opening modality (e.g., axial rotation, lateral translation, rotation around a lateral axis, etc.). Accordingly, in some variations, the health improvement device 310 may be used in combination with suitable generic containers with the detectable opening modality, without the need for a specialized container.

Furthermore, the health improvement device 310 may be removably coupled to the medication container so as to be swappable among different containers sharing the same opening modality. For example, the health improvement device 310 may be removably coupled to the medication container with adhesive (e.g., adhesive tape) and/or suitable fasteners (e.g., hook and loop fasteners, screws, clips, straps, etc.). As another example as shown in FIG. 3B, the health improvement device 310 may include or be coupled to an elastic material 310′ that wraps at least partially around a portion (e.g., cap, body, or both) of the medication container, such as a cap cover, sleeve, band, strap, etc. In some variations, the elastic material may include silicone, or other suitable elastomeric material. The elastic material may be stretchable to fit a variety of shapes and sizes of medication containers, thereby enabling the health improvement device 310 to be compatible with a range of shapes and sizes of medication containers. In variations in which the health improvement device 310 may be configured to be removably coupled to a medication container, one health improvement device 310 may be re-used with different containers, such as throughout multiple prescription refills or for different kind of medication.

Generally, in some variations, the health improvement device may include one or more sensors for detecting at least one status (e.g., parameter) of the health improvement device and/or detecting one or more user interactions with the health improvement device.

For example, in some variations, the health improvement device may include one or more sensors for detecting a container state (e.g., opened or closed, or in the process of opening or closing, time open, time closed, movement, acceleration). For example, the sensor may include an accelerometer and/or a gyroscope (e.g., the sensor may include an inertial measurement unit, or IMU), and may be configured to detect position and/or orientation of the container lid. Accordingly, the sensor may be configured to detect one or more opening modalities, such as axial rotation. The sensor signal may be analyzed to determine a container state (e.g., opened or closed, or in the process of opening or closing). The container state may indicate when the patient has taken medication from the medication container, and/or is in the process of doing so.

One illustrative example of the health improvement device 310 is used with a medication container that is opened by axially rotating a threaded container lid relative to the container body. In this example, the processor may analyze the sensor signal to determine the total angular rotation to a predetermined threshold. Directionality of the rotation may also be considered. For example, assuming a right hand-threaded lid, if the detected total angular rotation in a counter-clockwise direction (when viewed from the top) exceeds a first predetermined threshold (e.g., at least 360 degrees, at least 720 degrees, at least 1080 degrees, etc.), this may indicate that the container has been opened. Furthermore, if the detected total angular rotation in a counter-clockwise direction is non-zero and does not exceed the first predetermined threshold, this may indicate that opening of the container is in progress. Similarly, the detected total angular rotation in a clockwise direction (when viewed from the top) when compared to a second predetermined threshold (which may be the same or different from the first predetermined threshold) may indicate that the container has been closed or closing of the container is in progress. Additionally or alternatively, other metrics such as angular velocity above a predetermined threshold for at least a predetermined period of time may be used to indicate container opening and/or closing. Thus, the sensor may be configured to generate a sensor signal indicating opening or closing of the medication container when the sensor is coupled to the medication container.

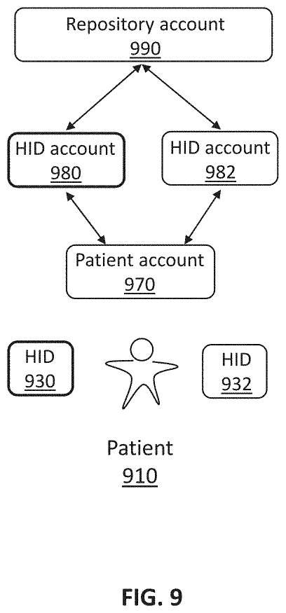

In some variations, the health improvement devices may be configured to substantially enclose or surround an existing medication container. In some variations, the health improvement include a medication monitor adapted to receive a medication container such as any standard sized prescription pill bottle. The health improvement device may be configured to gather medical-related data (e.g., through one or more sensors) and communicate this data to one or more of the patient, computing devices, and predetermined contacts. As shown in the block diagram of FIG. 10, in some variations, a health improvement device 1000 (e.g., medication monitor) may generally include an enclosure 1002 comprising a first sensor 1010, first transceiver 1020, first holder 1030, output device 1040, processor 1050, memory 1060, and/or power source 1070. In some variations, a lid 1004 may comprise one or more of a second sensor 1012, second transceiver 1022, and/or second holder 1032. The lid 1004 may be removably couplable to the enclosure 1002.

In some variations, the first and second holders 1030, 1032 may be configured to cooperatively position the medication container within a cavity of the medication monitor 1000. The first sensor 1010 and/or second sensor 1012 may be configured to generate medication data and one or more of the first and second transceivers 1020, 1022 may be configured to transmit at least the medication data to one or more computing devices. In some variations, the first sensor 1010 and/or second sensor 1012 may further include an accelerometer and/or a gyroscope configured to detect handling of the enclosure 1002 by the patient (e.g., opening or closing of the medication monitor, motion, acceleration).

In some variations, a health improvement device 1000 may comprise an enclosure 1002 comprising a first holder 1030 configured to position at least a portion of a medication container within a cavity of the enclosure 1030. For example, the first holder 1030 may be an adjustable holder configured to position at least the portion of the medication container within the cavity of the enclosure. The medication container may, for example, have a set of dimensions (e.g., diameter, height, cross-sectional shape, etc.) within a predetermined range of dimensions. For example, first holder 1030 may adjustably position and hold any number of medication container form factors, which may be predetermined standard form factors of medication containers as dispensed by a health care professional. Exemplary variations of the first holder 1030 for accommodating a range of dimensions are described in further detail below. In some variations, the first and second holders 1030, 1032 may be configured to adjustably position the medication container within the cavity of the medication monitor 1000. However, in other variations, the medication monitor may include only one holder, such as only a first holder 1030 coupled to the enclosure 1002, or only a second holder 1032 coupled to the lid 1004. In this manner, the health improvement device 1000 may be compatible with a wide range of medication containers that may vary in shape and size.

A lid 1004 (or a portion thereof) may be removably couplable to the enclosure 1002 such that the health improvement device 1000 substantially surrounds the medication container wherein the lid 1004 is secured to the enclosure 1002, and permit access to contents of the medication container when the lid (or portion thereof) is uncoupled from the enclosure 1002. In some variations, the medication container is used without its own lid when placed in the medication monitor 1000. This allows the lid 1004 of the health improvement device 1000 to enclose and seal the medication container when the lid 1004 is attached to the enclosure 1002. That is, the lid 1004 may replace the lid of a medication container.

Moreover, a lid 1004 may be configured to open by axially rotating a threaded portion of the lid 1004 relative to the enclosure 1002 in a similar manner as described with respect to FIGS. 3A-3B. In this example, the processor 1050 may analyze the second sensor 1012 signal to determine the total angular rotation to a predetermined threshold. Directionality of the rotation may also be considered (e.g., to determine an open state, a closed state, or the motion of opening or closing of the medication monitor, etc.). For example, similar to the health improvement device 300 described above, assuming a right hand-threaded lid, if the detected total angular rotation of the lid in a counter-clockwise direction (when viewed from the top) exceeds a first predetermined threshold (e.g., at least 360 degrees, at least 720 degrees, at least 1080 degrees, etc.), this may indicate that the medication monitor 1000 (and/or medication container) has been opened. Furthermore, if the detected total angular rotation in a counter-clockwise direction is non-zero and does not exceed the first predetermined threshold, this may indicate that opening of the medication monitor 1000.

In some variations, at least a portion (e.g., sidewall) of the enclosure 1002 may comprise a transparent portion (e.g., window, or may be made of transparent material) configured to allow a patient to view at least a portion of the medication container within the medication monitor. Furthermore, the medication container may be positioned against or proximate to the transparent portion (such that the medication container is asymmetric with respect to the enclosure 1002. Similarly, additionally or alternatively, at least a portion of the lid 1004 may comprise a transparent portion. Through such transparent portions, one or more characteristics of the medication container (e.g., medication name, medication dosage, medication quantity, treatment plan instructions, patient name, prescribing physician information, expiration date, and/or other information on a prescription label, etc.) may be viewable while the medication container is enclosed or surrounded by the medication monitor 1000. Access to this information may, for example, enable manual or automatic (e.g., through optical character recognition or other computer vision techniques) verification of a medication treatment plan, and may enable automatic reading of medication-related information which may be provided to a computing device as described in further detail below for encouraging patient behavior.

In some variations, the medication monitor 1000 may comprise an optical sensor. In some of these variations, the optical sensor may be configured to image one or more portions of the medication container (e.g., medication label) as medication data. One or more of the processor 1050 and computing device may be configured to process an image of a medication label to identify the medication within the medication container. For example, the medication data may be processed using an optical character recognition (OCR) process. The optical sensor may be disposed on any surface of the medication monitor 1000 including an inner and outer surface of the enclosure 1002 and lid 1004.

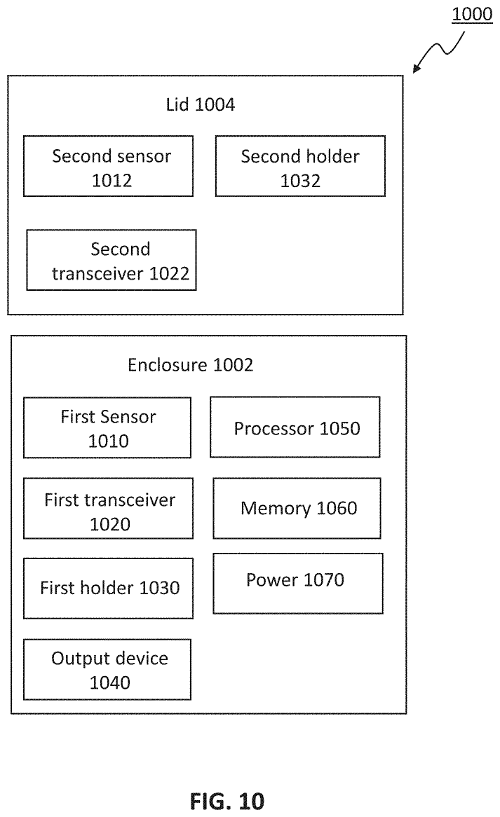

FIG. 11 is an illustrative schematic of an exemplary variation of a health improvement device similar to health improvement device 1000. In this variation, the health improvement device includes a medication monitor 1100 that may comprise a lid 1120 removably couplable to an enclosure 1110. A medication container 1130 may be positioned within a cavity of an enclosure 1110 such that the medication monitor 1100 may substantially surround the medication container 1130 when the lid 1120 is coupled to the enclosure 1110. For example, the enclosure 1110 and lid 1120 may comprise respective cavities configured to hold respective portions of the medication container 1130. Furthermore, the medication monitor 1100 may be configured to adjustably position the medication container within the cavity of the enclosure 1110 based on the dimensions of the medication container 1130. For example, the enclosure 1110 may include a first holder (not pictured) that positions a first portion (e.g., bottom) of the medication container 1130, while the lid 1120 may include a second holder (not pictured) that positions a second portion (e.g., lid or top portion) of the medication container 1130. The first and/or second holders may be universal or adjustable, such that the medication monitor 1100 may be configured as a one-size-fits-all enclosure 1110 for any medication container 1130 having a predetermined range of dimensions. This may increase adoption and usage of the medication monitor 1100 by a patient by allowing the patient to utilize, for example, any industry standard-sized medication containers with the methods and systems described herein. In some variations, the medication container 1130 without its lid is simply placed within the medication monitor to add the functionality of the medication monitor 1100. When another medication container is received (e.g., a new bottle), the medication monitor 1100 may be re-used with the previous medication container being replaced. Although the medication container 1130 shown in FIG. 11 is generally cylindrical, the medication container 1130 is not so limited and may comprise any predetermined shape and dimension used to hold medication. Some bottles may be cylindrical while others may be generally cuboidal (e.g., rectangular prism). Accordingly, the medication monitor 1100 may adjust to accommodate such varied shapes and sizes of medication container.

In some variations, a holder 1030, 1032 as described here may be configured to fix the position of a medication container relative to the enclosure 1002. For example, the holder 1030, 1032 may generally position the medication container such that the medication container and the medication monitor are similarly oriented (e.g., when disposed within the medication monitor 1000, the central longitudinal axes of the medication container and enclosure and/or lid may be substantially aligned, coincident, or parallel. This may allow a patient to obtain medication from the medication monitor 1000 in substantially the same manner as medication obtained from the medication container (e.g., patient uncouples the lid 1004 from the enclosure 1002 and tips over the enclosure 1002 such that medication is dispensed). In some variations, the medication container may be positioned at a desired orientation and location relative to the enclosure 1002 to aid dispensation of medication. For example, the medication container may be disposed at a center of the enclosure 1002, asymmetrically within the cavity of the enclosure 1002, against a sidewall of the enclosure 1002, parallel to an opening of the enclosure 1002, combinations thereof, and the like.

As described in further detail below, a holder 1030, 1032 may be adjustable to position a medication container relative to a bottom, sidewall, and/or top of the medication monitor 1000, and may further secure the medication container within the medication monitor 1000. In some variations, a holder 1030, 1032 may comprise one or more structures and/or mechanisms configured to position a medication container relative to an enclosure 1002 (and/or relative to the lid).

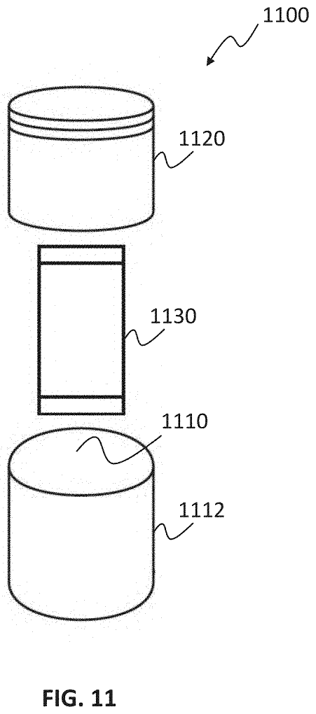

For example, as shown in FIGS. 12A and 12B, a holder 1230 may comprise a compressible member configured to transition between an expanded configuration and a compressed configuration. For example, the compressible member may comprise a foam material (e.g., open cell foam, closed cell foam) configured to compress between the enclosure 1220 (and/or lid) and a medication container 1210 disposed therein. The compressible member may, for example, be arranged circumferentially or around an inner perimeter of the enclosure and/or lid as shown in FIGS. 12A and 12B. The compressible member may be configured to transition from the expanded configuration (FIG. 12A) to the compressed configuration (FIG. 12B) in response to placement of the medication container into the cavity of the enclosure. The compressible member may be configured to surround at least a portion of the medication container in the compressed configuration, thereby allowing the medication container to fit snugly in the enclosure or other portion of the medication monitor as shown in FIG. 12B. For example, the compressible member may be disposed on one or more of a bottom surface and sidewall of the enclosure 1002. In some of these variations, the compressible member may be elastic.

In some variations, a holder may comprise an expandable member configured to transition between a deflated configuration and an expanded configuration. For example, the expandable member may comprise an inflatable member, balloon, pouch, bag, and the like. The expandable member may, for example, be arranged circumferentially or around an inner perimeter of the enclosure and/or lid. The expandable member is configured to transition from the deflated configuration to the expanded configuration (or vice versa) to position at least the portion of the medication container within the cavity of the enclosure and/or relative to the lid. The expandable member in the expanded configuration or deflated configuration may form a predetermined shape (e.g., inverse or complementary shape of a medication container, such as to snugly receive the medication container). The expandable member in the expanded configuration may be partially inflated in order to allow the expandable member to conform to medication containers of different shapes and/or sizes within the cavity of the enclosure and/or relative to the lid. For example, a medication container having a narrow lower portion and a wide upper portion may deform the inflatable member such that the expandable member in the expanded configuration has a wide lower portion and a narrow upper portion. In some variations, the expandable member may be configured to receive a fluid comprising one or more of liquid (e.g., water, saline) and gas (e.g., air, nitrogen).

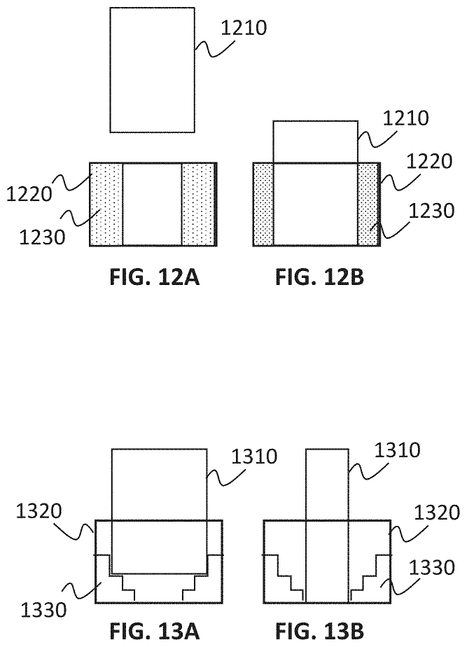

In some variations, a holder may comprise one or more structures within the cavity of the enclosure configured to provide a friction fit against a medication container. For example, as shown in FIGS. 13A and 13B, a holder 1330 may comprise a set of steps increasing in height toward a perimeter of the enclosure 1002 (e.g., concentric stepped rings). This may allow placement of medication containers 1310 with different diameters (e.g., larger diameter as shown in FIG. 13A, smaller diameter as shown in FIG. 13B) to be placed securely into the cavity of the enclosure 1320.

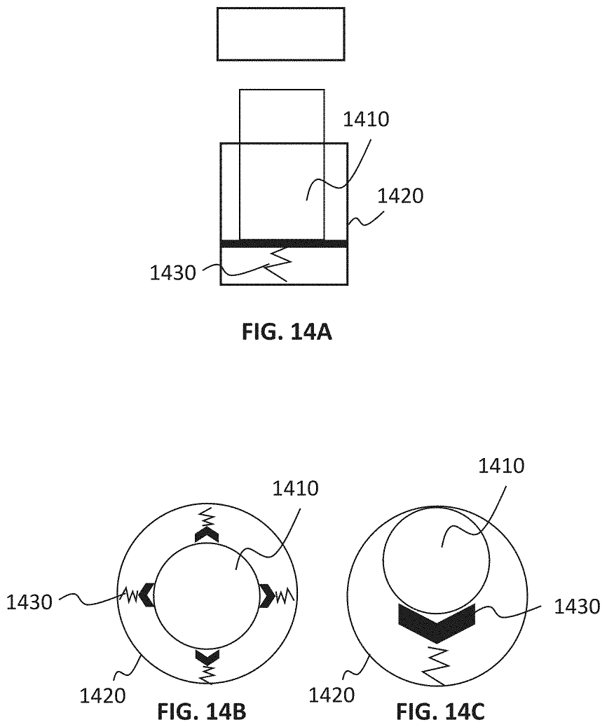

In some variations, a holder may comprise a set of one or more springs. For example, as shown in FIG. 14A, one or more springs 1430 parallel to a longitudinal axis of the enclosure 1420 may be configured to control a depth of a medication container 1410 within the enclosure. For example, one or more springs 1430 may contact an end surface (e.g., top, bottom) of the medication container. Additionally or alternatively, as shown in FIGS. 14B and 14C, one or more 1430 springs may be disposed around a circumference or inner perimeter of the medication container. In some variations, the one or more springs may be radially distributed so as to generally center the medication container 1410 within the enclosure 1420 (FIG. 14B). In other variations, one or more springs may be configured to urge the medication container a sidewall of the enclosure 1420 (FIG. 14C). Such springs may, for example, be coupled to a paddle or other container surface-engaging feature to brace against the medication container. The springs may be compression springs or other suitable spring-like mechanisms.

In some variations, a holder may comprise a combination of the features described above. For example, the lid 1004 may comprise a compressible material to form a seal around an opening of the medication container and the enclosure 1002 may comprise an inflated pouch configured to hold the medication container within a center of the enclosure 1002.

In some variations, a holder may comprise a mechanical actuator configured to position the medication container within a cavity of the enclosure 1002. For example, the actuator may comprise a plunger, lever, switch, combinations thereof, and the like.

In some variations, the medication monitor includes at least one sensor (e.g., the first sensor 1010 and/or the second sensor 1012) that may be configured to generate a sensor signal indicating medication-related data associated with a patient (e.g., medication data), and at least one output device (e.g., display, speaker) configured to provide feedback to the patient in response to the sensor signal. One or more of the enclosure 1002 and lid 1004 may comprise the transceiver 1020, 1022 (or transmitter) for communicating the medical-related data to another computing device, such as a personal computing device (e.g., mobile phone or tablet, etc.). A processor 1050 may be configured to receive a sensor signal from one or more of the first and second sensors 1010, 1012 and coordinate operation of other components such as the output device 1040 and/or transceiver 1020, 1022. Furthermore, the health improvement device 1000 may include one or more power sources 1070, such as a battery, which may be used to provide electrical power to the processor 1070, sensors 1010, 1012, and any other suitable electrical components. Additionally or alternatively, the lid 1004 may comprise one or more of the output device 1040, processor 1050, memory 1060, and power 1070.

### Medication Estimation

In some variations, the health improvement device 200 may include one or more sensors to detect a change in medication content (e.g., change in fluid volume, change in pill count) within the medication container, as a change in container state. For example, in one variation, the health improvement device may include one or more capacitive sensors configured to measure change in capacitance of the medication content. One or more capacitive sensors may, for example, be coupled to a surface of the medication container (e.g., on a bottom or base of the container body, on a sidewall of the container body, on a top surface of the container body, and/or on an underside of the container cap, etc.) and be capacitively coupled to the contents of the container. The one or more capacitive sensors may be coupled to an internal surface of the container, or to an external surface of the container (such as if the container material is made of a capacitive material. For example, a change (e.g., decrement) of capacitance exceeding a predetermined threshold may indicate that a medication dose has been taken. Examples of detecting a change in medication content based on capacitance are described in further detail below, and may be incorporated into variations of the health improvement device such as health improvement device 310. In another variation, the health improvement device 310 may include one or more force or pressure sensors configured to measure change in weight of the medication content. One or more force or pressure sensors may, for example, be coupled to a lower internal or external surface of the medication container. For example, a change (e.g., a decrement) of force or pressure exceeding a predetermined threshold may indicate that a medication dose has been taken.

In some variations, it may be determined that a medication dose has been taken based at least in part on detecting an indication that the medication container has been opened and/or closed (or is in the process of being opened or closed) in combination with detecting an indication of a change in medication content within the container (e.g., due to change in capacitance or weight of the medication content). For example, in some variations, it may be determined that a medication dose has been taken only if the health information device detects both that the medication container has been opened and that there is a sufficient decrement in medication content.

In some variations, one or more sensors 220 may be configured to generate medication data indicating a quantity of medication and/or a change in medication quantity (e.g., change in fluid volume, change in pill count) within a medication container. For example, the sensor may be configured to count the number of pills or a volume of liquid medication within the medication container. For example, in one variation, the medication container may include one or more capacitive sensors. One or more capacitive sensors may, for example, be coupled to a surface of the medication container (e.g., on a bottom or base of the container, on a sidewall of the container, on a top surface of the container, and/or on an underside of a lid on the container, etc.) and be capacitively coupled to the contents of the medication container. Similarly, with respect to the variation described above with respect to FIG. 11, a medication monitor 1000 may include one or more capacitive sensors. One or more capacitive sensors may, for example, be coupled to a surface of the medication monitor 1000 (e.g., on a bottom or base of the enclosure 1002, on a sidewall of the enclosure 1002, on a top surface of the enclosure 1002, and/or on an underside of the lid 1004, etc.) and be capacitively coupled to the contents of the medication container. In some variations, the one or more capacitive sensors may include an FDC2214 sensor, an LC resonator, or other suitable sensor. In these variations, a change (e.g., decrement) of capacitance exceeding a predetermined threshold may indicate that a medication dose has been taken. In some variations, a change in capacitance must be present for at least a predetermined threshold duration (e.g., 5 seconds, 10 seconds) in order to be considered as an indication that a medication dose has been taken. Furthermore, the magnitude of change of capacitance may be correlated to an estimated dosage amount that has been taken.

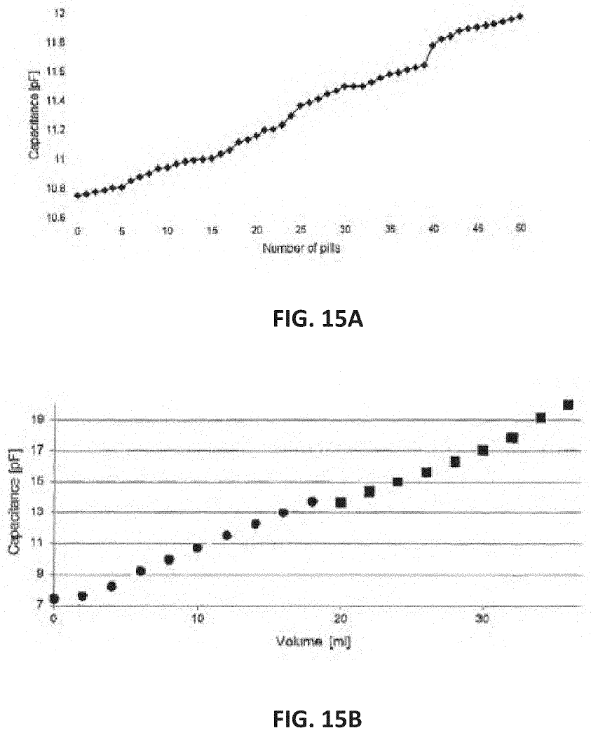

In some variations, the capacitance measurements and/or change in capacitance measurements may be correlated to medication quantity removed from the container via lookup tables, curves, formulas, and the like. For example, FIG. 15A illustrates an empirical plot illustrating an exemplary relationship between a number of pills present in a container and resulting capacitance measurement. As another example, FIG. 15B illustrates an empirical plot illustrating an exemplary relationship between volume of medication present in a container and resulting capacitance measurement.

In another variation, the medication container (medication monitor 1000) may include one or more force or pressure sensors configured to measure change in weight of the medication. One or more force or pressure sensors may, for example, be coupled to a lower internal or external surface of the container or medication monitor. For example, a change (e.g., a decrement) of force or pressure exceeding a predetermined threshold may indicate that a medication dose has been taken.

### Output Device(s)

The output device(s) of the health improvement device may be configured to provide feedback to the patient in response to one or more sensor signals. Additionally or alternatively, feedback may be provided by an output device on a personal computing device (e.g., mobile phone) in response to medical-related data such as container state being transmitted to the computing device. In some variations, the feedback may include state of the health improvement device, such as a state of the container being open or closed. Suitable output devices include, for example, a display or an audio device, or other device for communicating with the patient through visual, auditory, tactile, and/or other senses. In some variations, the display may include, for example, at least one of a light emitting diode (LED), liquid crystal display (LCD), electroluminescent display (ELD), plasma display panel (PDP), thin film transistor (TFT), organic light emitting diodes (OLED), electronica paper/e-ink display, laser display, holographic display, or any suitable kind of display device. In some variations, an audio device may include at least one of a speaker, a piezoelectric audio device, magnetostrictive speaker, and/or digital speaker. Other output devices may include, for example, a vibration motor to provide vibrational feedback to the patient.

For example, the display may provide a visual graphic and/or message that includes feedback to the user about the state of the container (e.g., a graphic or animation representing an open container and/or a text message of “open” may be displayed). In another variation, the health improvement device may include auditory feedback such as an auditory message (e.g., the speaker may output the phrase “the container is open” or similar notification, and/or output a particular audio tone, pattern, or music correlated with the state of the container). Accordingly, the output devices may provide feedback that confirms container state or other medical-related data (e.g., estimated remaining medication content in the medication content). Furthermore, in some variations, the output device may provide other suitable information, such as an advertisement or promotional material that is displayed on a display or played on a speaker (e.g., for a short period of time, such as between about 3 seconds and about 5 seconds, about 4 seconds, or any suitable duration). An advertisement may be provided, for example, in response to detected user attention (e.g., detected opening or closing of a medication container to which the display is attached).

In some variations, the feedback may provide the patient with encouragement through the output devices. Such encouragement may be explicit to the user. For example, a display and/or audio device may output the phrase “Great job!” or similar encouraging language in response to the container being opened and/or closed. In other examples, a display may output a smiley face or similar happy graphic or animation, or a speaker may output a joyful tune or series of tones.

Additionally or alternatively, feedback may comprise instructions or other guidance related to the medication. For example, a reminder that the medication must be taken with food may be output by a display and/or a speaker. Furthermore, other communications generated by the mobile application may be output by the output device(s) of the health improvement device 310 and/or personal computing device. For example, the feedback may comprise one or more reminders based on a predetermined dosage schedule. For example, a reminder may indicate a time to take medication. In one variation, the output device may initially generate haptic feedback at a predetermined time if the health improvement device determines that the patient has not opened the medication container during a prescribed dosage time period (e.g., 8:00 a.m. to 10:00 a.m.) and a quantity of medication has not decreased based on sensor measurements such as those described above. If there is no change after a first predetermined time period (e.g., 30 minutes), the output device may generate a voice prompt encouraging the patient to take their prescribed dosage and display a corresponding prompt on the display, and/or through an associated user interface on a mobile computing device. For example, the prompt may include encouragement to comply with a dosage schedule. If there is no change after a second predetermined time period (e.g., 60 minutes), the output device may generate an additional prompt (same or different from the first prompt) and send a notification to a predetermined contact. The predetermined contact may receive the notification through any communication protocol (e.g., SMS) on a computing device such as a mobile phone or personal computer through a web-based portal.

In some variations, the predetermined contact may be prompted to record audio and/or video feedback encouraging or reminding the patient to comply with the dosage schedule. A transceiver 250 in the health improvement device may receive the feedback and the output device may output the feedback to the patient. For example, the output device may output the received feedback to the patient in response to one or more sensors generating signal data corresponding to patient handling of the medication container. For example, after an accelerometer of the health improvement device 200 may generate a signal indicating that the patient has picked up the device 200, the encouraging feedback may be displayed to the patient on the output device (e.g., display 230 or speaker 240). Additionally or alternatively, the encouraging feedback may be displayed to the patient on an associated user interface on a mobile computing device.

In some variations, the feedback may comprise one or more warnings based on a predetermined dosage schedule (e.g., dosage alert). For example, a warning may be generated by the health improvement device 200 in response to medication data corresponding to a patient accessing the medication within a predetermined time period prior to a scheduled dosage time. The warning (e.g., double dose alert) may indicate that it is too early to consume the next dose of medication and may prompt the patient to wait until the next scheduled dosage time. For example, the warning may be generated and provided in response to a determination that the medication container has been picked up and/or opened more than expected number of times for a predetermined period of time (e.g., a double dose alert may be generated based on one or more sensor signals indicating an opening of the lid and/or handling of the medication monitor within a predetermined period of time after the user is determined to take a dose of medication, or indicating an opening of the lid multiple times within a predetermined time period). As another example, a warning may be generated by the health improvement device 200 in respo nse to medication data corresponding to a change in medication quantity outside a predetermined range for a predetermined dosage schedule. That is, a warning (e.g., incorrect dosage alert) may indicate that the patient has dispensed an incorrect amount of medication (e.g., overdose, underdose) from the health improvement device 200. The warning may further prompt the patient to correct the dosage dispensed (e.g. “Please take 5 ml more of medication”, “Please return 3 pills to the bottle”).

The output device may be configured to output the feedback to the patient and prompt the patient to comply with their dosage schedule. In some variations, one or more predetermined contacts may be notified of the warning based at least on the medication data. For example, a predetermined health care professional may receive a warning that the patient has potentially exceeded their dosage (based on time and/or quantity) and be prompted to generate feedback to the patient to encourage compliance with their dosage schedule. The output device may output the feedback to the patient.

In some variations, the health improvement device 200 may comprise a geolocator such as Global Positioning System (GPS) configured to generate position data of the health improvement device. The position data may be transmitted from the health improvement device to one or more computing devices. For example, a computing device associated with the patient may be configured to output the position data to an authorized user such as a patient and predetermined contact. In some variations, the output device may be configured to generate feedback comprising audio (e.g., a set of beeps) used to locate the medication monitor in response to instructions from the computing device. As such, the patient may follow the audio cues output by the medication monitor to locate their medication. As another example, the patient may follow a map or other visual guidance displayed on a computing device.

In some variations, the medical-related data may comprise medication data from a plurality of health improvement devices. The medication data may comprise a plurality of medications. The health improviement devices and computing device may, for example, be configured to process the medication data to generate interaction data comprising risks and side effects associated with medication interactions. One or more of the output device and computing device may be configured to output the interaction data. In some variations, the medication may be identified based on dosage data from a database associated with the National Library of Medicine database and other drug databases.

In yet other variations, encouragement may be provided in a subliminal manner to the user. For example, a positive affirmation or other encouraging messages may be modulated at a particular subliminal frequency to be understood subconsciously. Such positive affirmation modulated at a suitable frequency may retrain or reprogram the patient to feel better when taking medication. For example, in one illustrative variation, a positive affirmation may be modulated at a frequency of between about 9.5 Hz and 11.5 Hz (or about 10 Hz), or between about 5.0 Hz. and 6.0 Hz (or at about 5.5 Hz), to be understood by the patient at a subconscious level. The positive affirmation may then be output during the time that the medication container is determined to be open, such that the patient may associate a sense of well-being whenever he or she opens the medication container. Accordingly, such encouragement, when output for the patient to understand either consciously or subconsciously, may increase the likelihood that the user continue taking medication.

The transceiver (or transmitter) of the health improvement device may be configured to transmit medical-related data such as container data to one or more computing devices, such as a mobile phone. For example, container data may be based at least in part on the sensor signal (e.g., indicating an open state, a closed state, or in the process of opening or closing). In some variations, the transceiver may be configured to communicate with the computing device via a wired connection or a wireless connection such as with Bluetooth or other suitable communication protocol. In some variations, the transceiver may be configured to receive communication from the computing device, such as signals corresponding to instructions for the output devices.

It should be understood that a wide variety of health improvement devices may be used in the systems and methods described herein to encourage various types of patient behavior. Generally, health improvement devices may include one or more sensors configured to detect any suitable medical-related data associated with a patient. Examples of health improvement devices include, but are not limited to: a pedometer that quantifies steps or other activity level of a patient (e.g., wearable pedometer, a mobile device with pedometer feature, etc.), a glucose monitoring device that detects blood glucose levels of a patient or frequency of blood glucose testing, a blood pressure monitor that detects blood pressure of a patient, a sensor that tracks duration of sleep for a patient, and the like.

Each of these health improvement devices may include suitable output devices (e.g., display, speaker, etc.) providing visual, auditory, tactile, and/or other feedback to the patient, similar to that described above.

### Computing Device

Generally, the computing devices described herein may include a controller including a processor (e.g., CPU) and memory (which can include one or more computer-readable storage mediums). The processor may incorporate data received from memory and user input. The memory may include store instructions to cause the processor to execute modules, processes, and/or functions associated with the methods described herein. In some variations, the memory and processor may be implemented on a single chip, while in other variations they can be implanted on separate chips.

The processor may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions such as measurement data processing, measurement device control, communication, and/or device settings. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CDROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, a computing device may further include a communication interface configured to permit a patient and/or other used to control the computing device. The communication interface may include a network interface configured to connect the computing device to another system (e.g., Internet, remote server, database) by wired or wireless connection. In some variations, the computing device may be in communication with other devices via one or more wired or wireless networks. In some variations, the communication interface may include a radiofrequency receiver, transmitter, and/or optical (e.g., infrared) receiver and transmitter configured to communicate with one or more device and/or networks.

Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like). For example, devices (e.g., one or more computing devices and/or health improvement devices) may directly communicate with each other in pairwise connection (1:1 relationship), or in a hub-spoke or broadcasting connection (“one to many” or 1:m relationship). As another example, the devices (e.g., one or more computing devices and/or one or more health improvement devices, etc.) may communicate with each other through mesh networking connections (e.g., “many to many”, or m:m relationships), such as through Bluetooth mesh networking.

The communication interface may further include a user interface configured to permit a user (e.g., patient, health care professional, etc.) to control the computing device. The communication interface may permit a user to interact with and/or control a computing device directly and/or remotely. For example, a user interface of the computing device may include an input device for a user to input commands and an output device for a user to receive output (e.g., prompts on a display device).

### Network

In some variations, the systems and methods described herein may be in communication via, for example, one or more networks, each of which may be any type of wired network or wireless network. A wireless network may refer to any type of digital network that is not connected by cables of any kind. Examples of wireless communication in a wireless network include, but are not limited to, cellular, radio, satellite, and microwave communication. However, a wireless network may connect to a wired network in order to interface with the Internet, other carrier voice and data networks, business networks, and personal networks. A wire network may be carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN) like the Internet, and virtual private networks (VPN). “Network” may refer to any combination of wireless, wired, public, and private data networks that may be interconnected through the Internet to provide a unified networking and information access system. Furthermore, cellular communication may encompass technologies such as GSM, PCS, CDMA or GPRS, W-CDMA, EDGE or CDMA200, LTE, WiMAX, and 5G networking standards. Some wireless network deployments may combine networks from multiple cellular networks or use a mix of cellular, Wi-Fi, and satellite communication.

### GUI

As described above and as shown in FIG. 1, a health improvement device 120 may interface with a computing device 130, such as a mobile computing device. The computing device may be configured, for example, to execute a method 400 for encouraging patient behavior, by executing computer code in a software module. In some variations, the software module is in the form of a mobile application being executed by a mobile device (e.g., phone or tablet) and include a graphical user interface (GUI) for a patient or other user. Through interfacing with the health improvement device 120 and/or a patient, for example, the computing device may execute a method 400 for encouraging patient behavior.

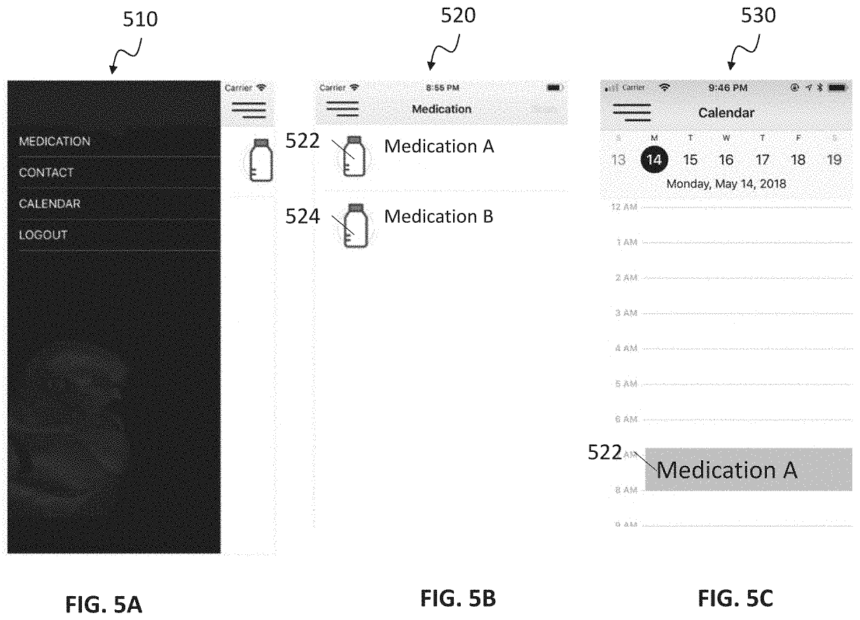

Generally, as shown in FIG. 4, a method 400 for encouraging patient behavior may include receiving a sensor signal 420 from a health improvement device, generating feedback to the patient based on the sensor signal 430, and providing the feedback to the patient 440. As described above, feedback may include visual, auditory, tactile, and/or other suitable feedback to the patient. In some variations, the method 400 for encouraging patient behavior may further include providing a reminder to the patient to engage in an action associated with a predetermined health regimen 410.

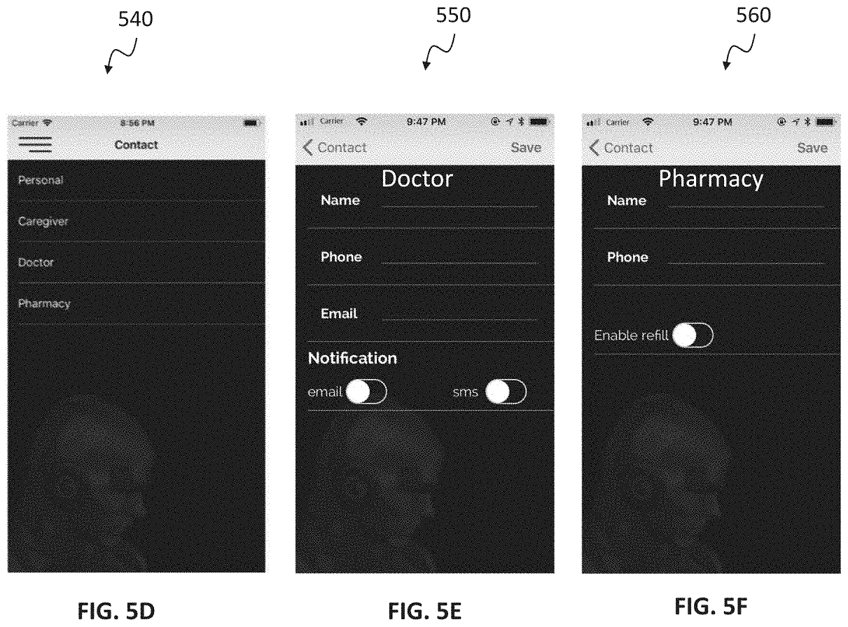

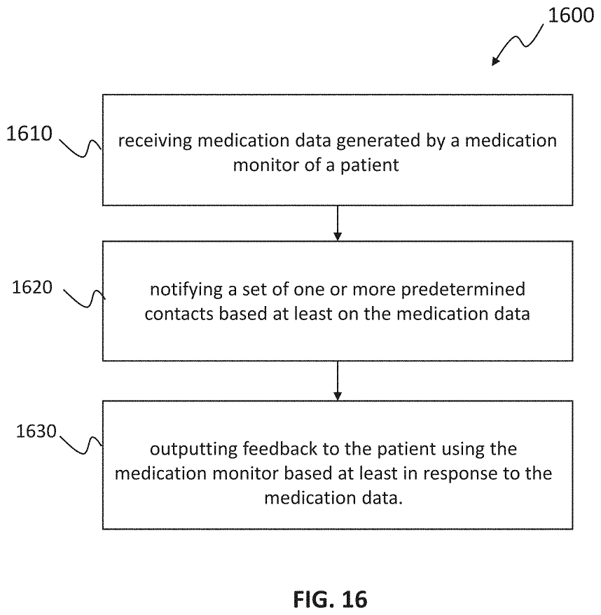

Exemplary variations of a GUI for encouraging patient behavior is shown in FIGS. 5A-5F. The GUI shown in FIGS. 5A-5F relate to a method for encouraging patient compliance with a predetermined medication plan and may, for example, be used in conjunction with the health improvement device 310 shown in FIGS. 3A, 3B, 16, and described above. The GUIs may be associated with a patient account that may aggregate information relating to one or more health improvement devices. The patient account may, for example, be secured with a username and password.

In some variations, one or more third party computing devices (e.g., for a healthcare professional, a family member, a friend, and/or other trusted contact) may be configured to display similar GUIs, so as to monitor and/or receive alerts regarding the patient's behavior, as further described below with respect to each GUI. Each trusted contact may have his or her own respective user account to access information relating to one or more patients. Information displayed on the third party computing devices may, for example, be received over a network in communication with the computing device of the patient such that third party computing devices may be synchronized with a patient's computing device.

FIG. 5A displays one variation of a GUI 510 including a user menu enabling access to various submenus including a selectable submenu for managing one or more medications, a selectable submenu for managing contact information for the patient and/or other parties, and a selectable submenu for managing a calendar for medication intake. The user menu may include a selectable option to login and/or logout, such as to enable toggling between different patient accounts and/or logging out to protect privacy.

FIG. 5B displays another variation of a GUI 520 summarizing a health regimen program associated with the patient. The program may include, for example, one or more entries each associated with a respective medication for the patient. Each entry may include a graphical representation, text description, and/or other suitable identifier for the medication. Additionally, each entry may be associated with a respective health improvement device coupled to the associated medication container. For example, a first medication entry may include a first representative icon 522 and a text label “Medication A”, and a first health improvement device (e.g., similar to health improvement device 310) may be coupled to at least a portion of the container for Medication A. Similarly, a second medication entry may include a second representative icon 524 and a text label “Medication B”, and a second health improvement device may be coupled to at least a portion of the container for Medication B.

In some variations, the representative icon 522 or 524 provides feedback to the patient based on a sensor signal from the associated health improvement device. For example, if the first health improvement device provides a sensor signal that the container for Medication A is being opened or has been opened, the appearance of the representative icon 522 may change to reflect this container status change. In one example, the color, size, and/or shape of the representative icon 522 to reflect the container status change. As another example, the representative icon 522 may become animated (e.g., become a blinking icon or include an animation of the container lid being removed from the container body). As another example, the representative icon 522 may be replaced with another icon (e.g., a green dot to indicate that the container is open). Similarly, text labels for Medication A may change according to the status of the container for Medication A (e.g., to read “Medication A—open” or the like).

In some variations, each entry in the health regimen program may be selectable, such that additional details about the entry may be entered, edited, and/or viewed. For example, selection of the entry for “Medication A” may result in the display of one or more fields (not shown) corresponding to identifying information such as name of medication, dosage amount, dosage frequency, special instructions (e.g., take with food, take on empty stomach), name and/or contact information of pharmacy supplying the medication, etc. At least some of such information may be manually entered, such as by the patient, through the GUI and the computing device's user input mechanisms (e.g., touchscreen). Additionally or alternatively, at least some of such information may be entered by scanning a label on the medication container, such as by using a camera equipped on the computing device that captures an image of the medication container label, and applying optical character recognition or other suitable machine vision techniques to parse label information into relevant fields of the GUI. As yet another example, at least some of such information may be prepopulated due to a data connection with another computing device (or server, database, etc.), such as that of a healthcare professional or pharmacy that enters the information relating to the patient's medication prescription and associating it with the health improvement device for that medication.

In variations of the GUI 520 that are accessible on third party computing devices, a similar set of entries associated with a patient's health regimen may be displayed. Appearance of at least a portion of an entry may change to provide feedback to a user, similar to that described above. For example, if a patient opens his or her medication container for Medication A that is outfitted with a health improvement device, the patient's computing device may receive a sensor signal indicating that the Medication A container is open. Following synchronization such as over a network, one or more third party computing devices may similarly reflect that the Medication A container is open. Accordingly, a trusted contact (e.g., healthcare professional, family member, friend, etc.) may be able to view that the patient has opened the Medication A container, thereby providing some measure of reassurance that the patient is taking the medication.

Although the GUI 520 of FIG. 5B is described in the context of the GUI being connected to health improvement devices (e.g., medication monitor) on medication containers to detect opening and closing of the containers and determine a quantity of medication, it should be understood that in other variations, the GUI 520 may summarize a health program including one or more entries associated with other kinds of health improvement devices. For example, a third entry (not shown) in the health program may be associated with a dosage schedule, blood glucose monitor, a pedometer, a blood pressure monitor, etc., and displayed in the GUI 520. In this manner, information relating to multiple health improvement devices for a patient may be aggregated in one place.

FIG. 5C displays another variation of a GUI 530 for managing a calendar in accordance with a health regimen. For example, the calendar may include timeslots for days and/or times to indicate when certain medication should be taken according to a health regimen. One or more reminders 522 may be calendared in a suitable timeslot, such that a notification may be provided to the patient at the beginning calendared timeslot, or at a predetermined period of time before the calendared timeslot (e.g., 30 minutes before, 15 minutes before, 5 minutes before, etc.). Such a notification may serve as a reminder and may be provided to the patient via a notification pop-up message, SMS text message, or the like. Similar to the medication information described above, the calendar information may be entered manually, populated after scanning and analyzing a medication label, and/or populated due to a data connection with another party.

In some variations, the notification may be adaptable. The notification may be automatically adaptable, or semi-automatically adaptable (e.g., a patient may be requested to approve and accept a suggested adapted notification). For example, if the health improvement device indicates that the container has been opened and/or closed before the calendared timeslot (e.g., within a predetermined period of time preceding the calendared timeslot, such as up to about 1 hour or about 30 minutes), the notification for that timeslot may be suspended or removed, on the assumption that the patient has taken the medication and does not require a reminder. Advantageously, such “smart” adaptable reminders may reduce unnecessary notifications to the patient. For example, if a patient is scheduled to take a medication dose around 9 AM and the patient elects to take the dose earlier, such as at 8:30 AM in view of a social engagement at 9 AM, a reminder for the 9 AM dose may be suspended so as to not bother the patient. Furthermore, if the health improvement device fails to indicate that the container has been opened and/or closed before the calendared timeslot, the notification for the timeslot may be replicated and/or repeated according to a predetermined time interval, on the assumption that the patient has not taken the medication (e.g., due to forgetfulness) and requires a reminder to do so.

As another example of an adaptable notification, the notification may be adapted from a first calendared timeslot to a second calendared timeslot based at least in part on patient routine. For example, using the GUI, a patient may set up notifications such that a notification for a morning medication dose occurs at 9 AM every day. However, if the health improvement device indicates that the patient tends to take the morning medication dose at a new time other than 9 AM, the notification may be adapted to occur at the new time. For example, the notification may be adapted if the health improvement device indicates that the container has been opened and/or closed at a different, non-calendared time (within a predetermined margin, such as 15 minutes) for at least a predetermined number of days in a row (e.g., at least 3 days, at least 5 days, etc.). As another example, the notification may be adapted if the health improvement device indicates that the container has been opened and/or closed at a different, non-calendared time (within a predetermined margin, such as 15 minutes) for at least a predetermined percentage of a certain number of days (e.g., at least 50% of the last n days).

In some variations, data from the patient computing device may be analyzed (e.g., with a cloud back-end or other server) to automatically adjust notifications. For example, the calendar of GUI 520 may be synchronized with another personal calendar of the patient, such that notifications may be adjusted in view of any time conflicts between calendared notifications and personal social engagements. For example, the system may detect that the patient has a meeting between 7 AM and 7:30 AM on a particular day which may make it difficult for the patient to take his or her medication dose that is calendared for 7 AM. Accordingly, the notification to take that medication dose may be adjusted to suitable time before or after the meeting (e.g., 6:45 AM or 7:45 AM), such that the patient need not be bothered during the work meeting. Furthermore, various machine learning algorithms or other suitable algorithms may be implemented to analyze a patient's overall schedule and identify suitable timeslots to remind the patient to take medication (e.g., the patient tends to have morning meetings at 9 AM on Monday, but at 10 AM on Tuesday, so a notification relating to a daily morning medication dose may be sent at 8:45 AM on Monday but 9 AM on Tuesday, to accommodate the patient's schedule).

In variations in which a GUI is provided on a third party computing device, a trusted contact may receive a notification in a similar manner. For example, if a health improvement device indicates that a patient has failed to take a medication dose in accordance with a calendared timeslot, a trusted contact may receive a notification pop-up message or SMS message indicating the same. This may, for example, allow the trusted contact to follow-up with the patient regarding the medication dose. Additionally or alternatively, if a health improvement device indicates that a patient has taken the medication dose, then the trusted contact may receive another notification serving as a confirmation of the same.

Other actions to be taken by the patient, such as a blood glucose testing event, blood pressure measurement event, exercise regimen, etc. may also be calendared and associated with respective notifications in a similar manner.

Another variation of a GUI 540 is shown in FIG. 5D. GUI 540 includes a list of selectable submenus that contain contact information. For example, GUI 540 may provide access to personal contact information for the patient, contact information for a caregiver or doctor (or other suitable healthcare professional), contact information for the patient's pharmacy, and/or the like. Personal contact information such as name, phone number, email address, etc. may be entered via a GUI (not shown). As another example, GUI 550 provides fields for information for a doctor such as name, phone number, and email. Other options relating to communication may additionally be provided, such as selectable options for doctor communications to be received over email or over SMS messages. As yet another example, GUI 560 provides fields for information for a pharmacy such as name of pharmacy, phone number, address, etc. Additionally other options relating to interactions with the pharmacy, such as enablement of triggering a refill of a medication prescription (e.g., after a predetermined number of times of opening and/or closing the medication container) by the pharmacy.

Accordingly, one or more GUIs may interface with one or more health improvement devices so as to encourage patient behavior, such as through a system of notifications or other reminders to encourage desired behavior.

Generally, as shown in FIG. 16, a method 1600 for encouraging patient behavior may include receiving medication data generated by a medication monitor of a patient using a computing device 1610, notifying a set of one or more predetermined contacts based at least on the medication data using the computing device 1620, and outputting feedback to the patient using the medication monitor based at least in response to the medication data 1630. As described above, feedback may include visual, auditory, tactile, and/or other suitable feedback to the patient. The feedback may comprise at least one prompt to modify the patient behavior. For example, the prompt may comprise encouragement to comply with a dosage schedule. The predetermined contacts may comprise one or more of a health care professional, a patient's partner, family member, and support group. In some variations, a communication channel may be established between the computing device and a health care professional device in response to the medication data. In some variations, the method 1600 for encouraging patient behavior may further include providing a reminder to the patient to engage in an action associated with a predetermined health regimen.

### Cryptocurrency Blockchain Environment

In some variations, the systems and methods described above may operate in a cryptocurrency blockchain environment that provides incentives to patients for engaging in certain health-improving behaviors. For example, patient behavior information may be communicated over a blockchain distributed network, and analyzed against one or more smart contracts (described in further detail below) defining token transactions to reward patient behavior, such as with a token within a cryptocurrency system.

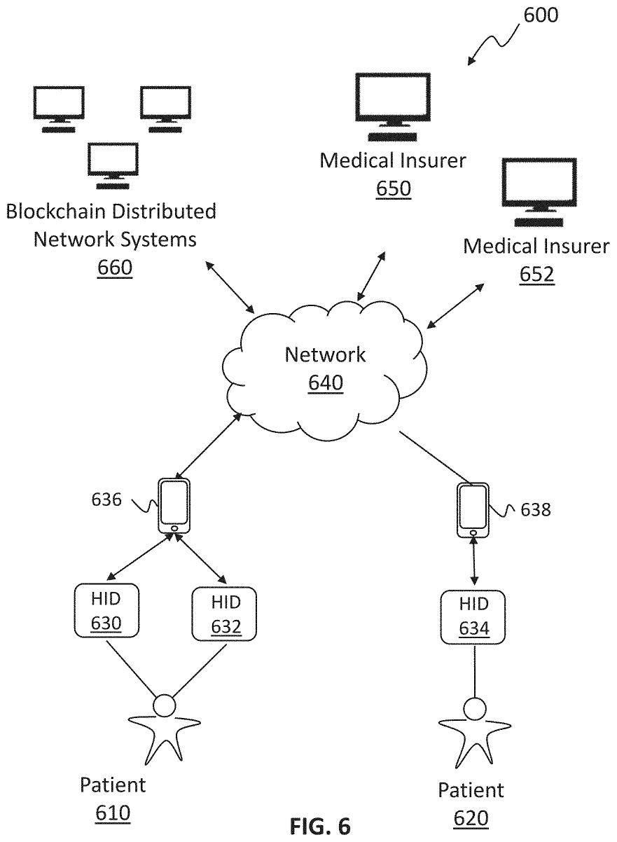

FIG. 6 depicts a block diagram illustrating an exemplary cryptocurrency blockchain program environment. The environment 600 may include a first patient 610 and/or a second patient 620 who are users participating in the cryptocurrency blockchain program environment. The first patient 610 and second patient 620 may be associated with a number of health improvement devices. For example, the first patient 610 may be associated with a first health improvement device 630 and a second health improvement device, where the first and second health improvement devices are configured to gather medical data related to the first patient. Similarly, the second patient 620 may be associated with a third health improvement device 634 configured to gather medical data related to the second patient. Examples of suitable health improvement devices include a device to detect opening and closing of a medication container, a pedometer, etc., similar to that described above.

Each of the health improvement devices may be in communication with a personal computing device, such as a personal computing device of the patient. For example, first and second health improvement devices 630 and 632 may be in communication with a first personal computing device 636 associated with the first patient, while the third health improvement device 634 may be in communication with a second personal computing device 638 associated with the second patient. As described above, a personal computing device may be, for example, a mobile phone. The personal computing devices 636 and 638 may be configured to communicate over a network 640 with the blockchain distributed network system 660 and, in some variations, one or more medical insurers 650 and 652 that may participate in the smart contract blockchain environment.

### Health Improvement Devices

Suitable health improvement devices participating in the cryptocurrency blockchain environment may, for example, gather medical-related data about a patient with one or more sensors, and communicate the data to a computing device, similar to that health improvement devices described above. For example, any of health improvement devices 630, 632, and 634 may include one or more sensors configured to detect the opening and/or closing of a medication container, thereby enabling monitoring of a patient taking medication. The health improvement devices may be in communication with a personal computing device that may be configured to provide feedback (e.g., notifications or other reminders) regarding a health regimen, similar to that described above.

### Blockchain Distributed Network Systems

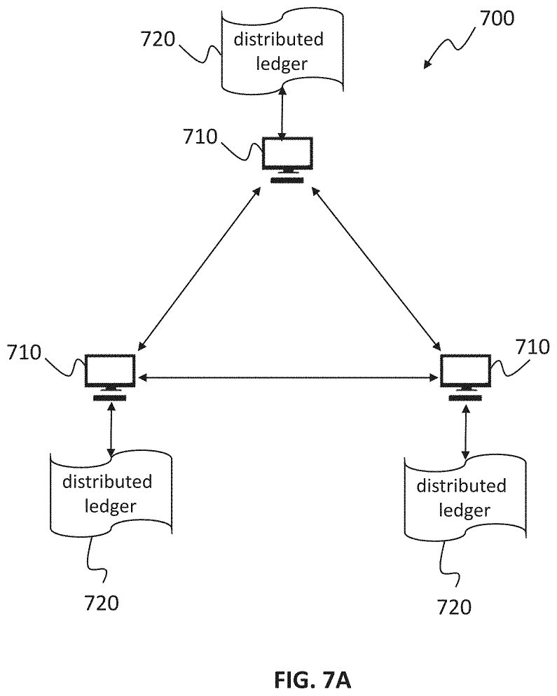

An illustrative schematic of a blockchain distributed network system 700 is shown in FIG. 7A. The blockchain distributed network system may be characterized by a decentralized configuration (e.g., may lack a central database and/or is not operated by a single administrator). A blockchain may be a distributed database that maintains a secure, substantially immutable list of data records across multiple “nodes” which are shown schematically as nodes 710 in FIG. 7A. The nodes are individual parts of the larger blockchain data structure. The nodes 710 are operably interconnected (e.g., via Internet), and each node may include, for example, a computer, database, or other suitable machine. The nodes 710 may be operated by multiple entities (e.g., each node by a different respective entity).

The nodes 710 collectively maintain a decentralized, distributed ledger 720 that may include a record of smart contract logic and/or set of transactions occurring across the network. Smart contracts are computer processes that digitally facilitate, verify, or enforce the negotiation or performance of a contract between entities. Smart contracts typically include logic emulating clauses in traditional contracts, except that the logic is partially or fully self-executing and/or self-enforcing once specified conditions are satisfied. Such smart contracts may be defined, for example, in a decentralized application including backend software code running on a decentralized peer-to-peer or blockchain distributed network. A record of transactions may include a record of exchange of data between different entities, such as across the network. Generally, for example, multiple entities may exchange money, tokens, contracts, medical records, or any other asset that may be described in digital form.

A node in the blockchain may have access to a complete or partial copy of the entire ledger 720. The data in the ledger may be organized in container data structures referred to herein as “blocks” arranged in sequence (e.g., according to order of transactions). As new transactions occur, they are added to the distributed ledger 720 as additional blocks. Each block may be uniquely identified by a cryptographic hash, which may be similar to a digital signature, and may refer to or otherwise be based on the previous hash in the block sequence. Accordingly, in some variations, a block may contain a block header (containing metadata such as a reference to the previous block's hash) and a set of transactions and/or other ledger data. The sequence of linked hashes may create a secure chain of information.

With reference to FIG. 7A, a transaction may be initiated at a node in the blockchain and communicated to other nodes in the blockchain such that all nodes have an updated copy of the entire ledger 720. At least some of the nodes may validate the transaction in accordance with a prescribed set of coded rules or requirements, before a new block may be added to the blockchain. Validation may be performed using a suitable validating process (e.g., proof of work, proof of stake, etc.) in order to verify the transaction. Accordingly, the distributed ledger may be updated across the blockchain distributed network to include the network's blockchain. In some variations, the blockchain distributed network system may update the distributed ledger periodically (e.g., every ten minutes).

There are several benefits to blockchain data structures. For example, by storing blocks of information that are identical across its network, the blockchain cannot be controlled by any single entity. This makes records stored on the blockchain easily verifiable and more robust against corruption (e.g., by a hacker) because modifying any information on the blockchain would require a large amount of computing power to override the entire network (and modify all copies of the ledger). Furthermore, the blockchain is immutable, allowing for a substantially permanent ledger of information.

As shown in FIG. 7B, a node in the blockchain distributed network, or a blockchain network system 702, may include at least one network communication interface 710, at least one processing device 730, and at least one memory device 740. The processing device 730 may be communicatively coupled to the network communication interface 730 and the memory device 750. In some variations, the memory device 750 may be configured to store a distributed ledger 720, which includes smart contract logic and rules, as well as transactions, as described above. For example, as shown in FIG. 7B, the distributed ledger 720 may include a first smart contract A with associated logic and rules, a second smart contract B with associate logic and rules, etc. In some variations, the distributed ledger 720 may operate with suitable application software code that is configured to instruct the processing device 740 to operate the network communication interface 730 as described herein. For example, the processing device 740 may be configured to utilize the network communication interface to obtain data (e.g., corresponding to transactions or other updates to the distributed ledger 720), such as from other suitable data sources such as other blockchain network systems interconnected via the blockchain distributed network. The processing device may store the obtained data in the system's copy of the distributed ledger 720 in the memory device 750.

### Cryptocurrency

Generally, the cryptocurrency blockchain environment may facilitate transactions of specialized tokens or cryptocurrency, that are transferred, validated, and tracked in the blockchain environment, such as in the manner described above. Upon an initial coin offering (ICO), an initial repository may be created and initialized with a certain number of tokens deposited for further use. For example, some of these tokens may be distributed among one or more health improvement device accounts, and may be further transferred or exchanged among entities in the blockchain environment, as further described below.

### Method in a Cryptocurrency Blockchain Environment

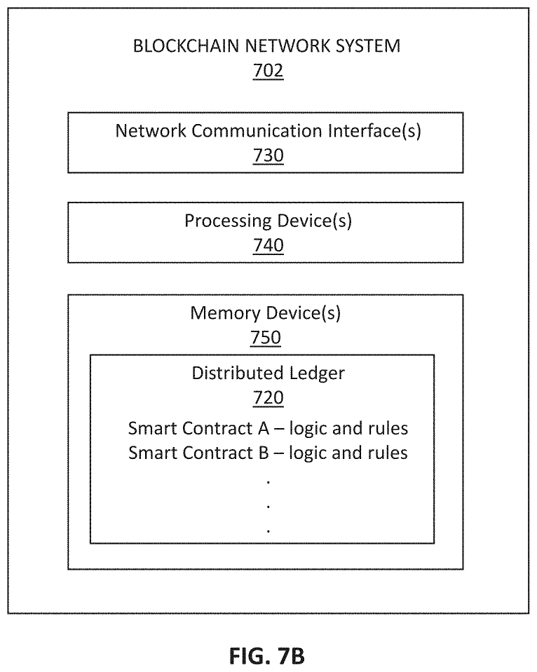

As shown in FIG. 8, a method 800 for using a blockchain distributed network for encouraging patient behavior may include receiving a patient behavior record over the blockchain distributed network 810, accessing a distributed ledger stored in a memory device 820 wherein the distributed ledger comprises a record of at least one smart contract associated with the patient behavior record, determining whether the patient behavior record satisfies at least one condition of the smart contract 830, instructing the transfer of one or more tokens to a patient account associated with the patient 840 if the at least one condition of the smart contract is satisfied, and updating the distributed ledger 850 based on the transfer of one or more tokens, through communication over the blockchain distributed network. One or more of these processes may, for example, be performed by a blockchain network system similar to that described above with respect to FIG. 7B.

A patient behavior record received at 810 may include medical-related data for a patient including sensor data from one or more health improvement devices, or information derived from such sensor data. For example, the patient behavior record may include information relating to compliance with a prescribed medication regimen, compliance with frequency of blood glucose testing, patient activity levels, etc. In some variations, the patient behavior record may be provided by a personal computing device. For example, with reference to FIG. 6, a personal computing device 636 may collect medical-related data from health improvement devices 630 and 632, then communicate the medical-related data over the network 640 to one or more blockchain network systems 660. In some variations, the personal computing device 636 may aggregate medical-related data before communicating it over the network 640 to one or more blockchain network systems 660. Additionally or alternatively, the medical-related data may be communicated periodically or intermittently (e.g., daily, weekly, bi-weekly, bi-monthly, monthly, etc.). Alternatively, in some variations, the patient behavior record may be provided directly from a health improvement device over the network to one or more blockchain network systems.

A distributed ledger may be accessed at 820, where the distributed ledger may include at least one smart contract stored in a memory device. The smart contract may include a set of one or more conditions relating to desired characteristics or qualities of the patient behavior record. For example, in the exemplary application of encouraging patient compliance with a medication regimen, the smart contract may include a condition that the patient took his or her medication generally on time at the most recent scheduled dosage time. As another example, the smart contract may include a condition that the patient consistently took his or her medication generally on time for a predetermined number of scheduled dosage times (e.g., for the most recent two, three, four, five, or any suitable number of recent scheduled dosage times) and/or for at least a predetermined percentage of the scheduled dosage times. However, other suitable smart contract conditions may be included. It should be understood that different aspects of the patient behavior health record may be compared against different smart contract conditions. For example, a first medication may be associated with a first smart contract condition and a second medication may be associated with a second smart contract condition different from the first smart contract condition, where the first and second medication may be part of the same patient behavior record.

Satisfaction of the smart contract condition may be determined at 830. For example, the patient behavior record may be compared against the smart contract condition to determine whether the smart contract condition is satisfied. If the smart contract condition is satisfied, other actions may be taken automatically in accordance with the logic and rules of the smart contract. It should be understood that in variations in which the distributed ledger includes multiple smart contracts, the patient behavior record need not satisfy all smart contract conditions.

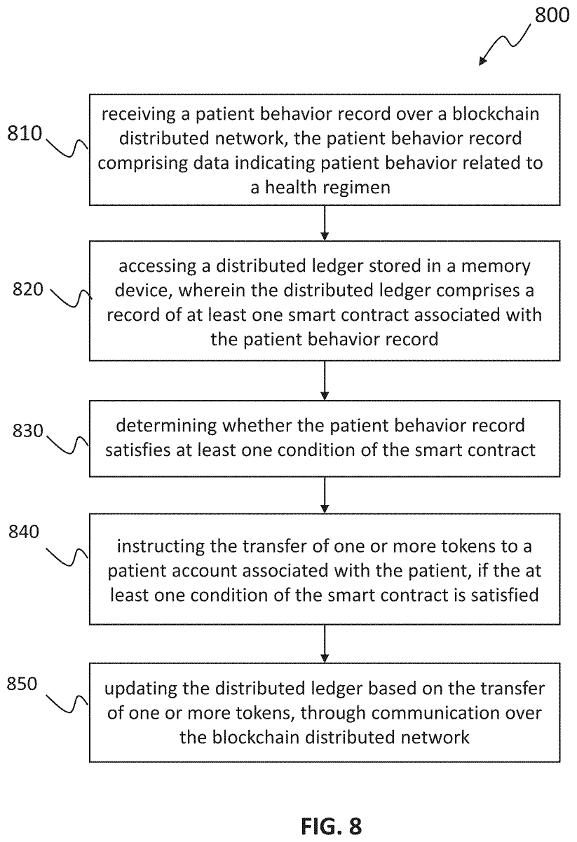

In response to determining that the patient behavior record satisfies at least one smart contract condition, the transfer of one or more tokens may be instructed at 840. The transfer may between two or more currency accounts. For example, as shown in FIG. 9, one or more health improvement device accounts (e.g., 980 and 982) may be connected to a patient account 970 associated with the patient 910. A respective health improvement device may be associated with each health improvement device account. For example, a first health improvement device 930 may be associated with a first health improvement device account 980, while a second health improvement device 932 may be associated with a second health improvement device account 982. A first smart contract condition may be related to patient behavior that is measured or otherwise indicated by the first health improvement device 930. If the first smart contract condition is satisfied, then at least one token may be transferred from the first health improvement device account 980 to the patient account 970. Similarly, a second smart contract condition may be related to patient behavior that is measured or otherwise indicated by the second health improvement device 932. If the second smart contract condition is satisfied, then at least one token may be transferred from the second health improvement device account 982 to the patient account 980.

Accordingly, patient behavior related to (e.g., as measured by) a particular health improvement device may be rewarded by a transfer of tokens from the account associated with that health improvement device into the patient account. The extent or rate of token transfer to the patient may vary among different patients (e.g., for the kind of patient behavior, more tokens may be transferred to a high-risk patient than for a low-risk patient, to thereby more strongly incentivize the high-risk patient.). In some variations, transfer of tokens may be aggregated and transferred in batches (e.g., daily, weekly, bi-weekly, bi-monthly, monthly, etc.). It should be understood that fewer (e.g., one) or more (e.g., three, four, five, etc.) health improvement device accounts may be in communication with a patient account as suitable (e.g., depending on the number of health improvement devices that the patient uses), such that the patient account may aggregate all tokens from multiple accounts in a “wallet.”

The patient may access his or her tokens on, for example, a GUI on his or her personal computing device that is configured to operate in the blockchain network. Transactions involving the transfer of tokens may be stored in the blockchain in an updated ledger at 850, providing a substantially immutable record of all transactions within the cryptocurrency blockchain environment.

In some variations, the health improvement device accounts may be replenished from the repository account 990. For example, the health improvement device accounts 980 and 982 may be replenished periodically, or whenever they contain a number of tokens below a predetermined threshold. Furthermore, alternatively, in some variations, the health improvement device accounts may be omitted, such that one or more repository account 990 may directly transfer one or more tokens from the repository account to the patient account 970.

In some variations, within the cryptocurrency blockchain system, the tokens may be applied toward benefits or rewards such as health-related benefits. For example, as shown in FIG. 6, in one exemplary variation, one or more medical insurers 650 may participate in the cryptocurrency blockchain environment 600. For example, medical insurers may have respective insurer accounts (not shown) that contain a suitable share of the tokens at any particular time. Such medical insurers may, offer a reduction or discount on health insurance premiums to a patient in exchange for tokens (e.g., in a transaction across the network 640). Accordingly, accumulation of tokens may be desirable by patients who would like to apply their tokens to reduce their insurance premiums.

In some variations, the medical insurers may offer health improvement devices to patients as part of a program to encourage patient participation in the cryptocurrency blockchain environment. Different smart contract conditions may exist on behalf of different medical insurers. For example, for the same patient behavior, the extent or rate of token transfer to the patient may vary for transactions performed on behalf of different medical insurers (and/or for different patients). Accordingly, patients may be encouraged to earn and accumulate tokens via compliance with health regimens, and such compliance may be characterized at least in part by the use of one or more heath improvement devices.

Thus, the systems and methods herein may be used to encourage particular patient behavior and lead to better health outcomes.

### EXAMPLES

Embodiment A1: A system for encouraging patient behavior, comprising: a sensor removably coupleable to a medication container and configured to generate a sensor signal indicating opening or closing of the medication container when the sensor is coupled to the medication container; an output device configured to provide feedback to a patient in response to the sensor signal; and a transceiver configured to transmit container data over a network to one or more computing devices, wherein the container data is based at least in part on the sensor signal.

Embodiment A2: The system of embodiment A1, wherein the output device comprises a display.

Embodiment A3: The system of embodiment A2, wherein the feedback comprises a visual graphic or message provided on the display.

Embodiment A4: The system of embodiment A1, wherein the output device comprises an audio device.

Embodiment A5: The system of embodiment A4, wherein the feedback comprises auditory feedback.

Embodiment A6. The system of embodiment A5, wherein the auditory feedback comprises an auditory message.

Embodiment A7. The system of embodiment A6, wherein the speaker is configured to output the auditory message at a frequency of about 10 Hz.

Embodiment A8. The system of claim A6, wherein the speaker is configured to output the auditory message at a frequency of about 5.5 Hz.

Embodiment B1: A method for encouraging patient behavior, comprising: receiving a sensor signal indicating opening or closing of a medication container, the sensor signal generated by a sensor removably coupleable to the medication container; generating feedback to the patient based on the sensor signal; providing the feedback to the patient at a mobile device associated with the patient.

Embodiment B2: The method of embodiment B1, wherein the feedback comprises a displayed graphic or message.

Embodiment B3: The method of embodiment B1, wherein the feedback comprises auditory information.

Embodiment B4. The method of embodiment B3, wherein providing the feedback comprises outputting an auditory message at a frequency of about 10 Hz.

Embodiment B5. The method of embodiment B3, wherein providing the feedback comprises outputting an auditory message at a frequency of about 5.5 Hz.

Embodiment B6. The method of embodiment B1, wherein generating feedback comprises comparing information derived from the sensor signal to a predetermined health regimen.

Embodiment B7. The method of embodiment B1, further comprising providing a reminder to the patient at the mobile device to engage in an action associated with a predetermined health regimen.

Embodiment C1: A method for using a blockchain distributed network for encouraging patient behavior, the method comprising: receiving a patient behavior record over the blockchain distributed network, the patient behavior record comprising data indicating patient behavior related to a health regimen; accessing a distributed ledger stored in a memory device, wherein the distributed ledger comprises a record of at least one smart contract associated with the patient behavior record; determining whether the patient behavior record satisfies at least one condition of the smart contract; instructing the transfer of one or more tokens to a patient account associated with the patient, if the at least one condition of the smart contract is satisfied; and updating the distributed ledger based on the transfer of one or more tokens, through communication over the blockchain distributed network.

Embodiment C2: The method of embodiment C1, wherein the patient behavior record comprises information indicating level of patient compliance with the health regimen.

Embodiment C3: The method of embodiment C2, wherein the patient behavior record comprises information indicating intake of medication in accordance with a treatment plan.

Embodiment C4: The method of embodiment C1, wherein the smart contract defines a token transaction in exchange for specified patient behavior.

Embodiment C5: The method of embodiment C1, wherein the transfer of one or more tokens is from a health improvement device account associated with a health improvement device.

Embodiment C6. The method of embodiment C5, wherein the health improvement device includes one or more sensors coupleable to a medication container.

Embodiment C7: The method of embodiment C5, wherein the health improvement device is a pedometer.

Embodiment C8: The method of embodiment C5, wherein the health improvement device is a glucose monitoring device.

Embodiment C9: The method of embodiment C5, wherein the health improvement device is a blood pressure monitor.

Embodiment C10: The method of embodiment C5, further comprising transferring one or more tokens from an insurer account associated with a medical insurance company to the health improvement device account.

Embodiment C11. The method of embodiment C10, wherein the one or more tokens is redeemable with the medical insurance company for a benefit.

Embodiment C12. The method of embodiment C1, wherein the one or more tokens is associated with a cryptocurrency.

Embodiment D1: A system for using a blockchain distributed network for encouraging patient behavior, the system comprising: a memory device; and a processing device operatively coupled to the memory device, wherein the processing device is configured to execute computer-readable code to: receive a patient behavior record over the blockchain distributed network, the patient behavior record comprising data indicating patient behavior related to a health regimen; access a distributed ledger stored in a memory device, wherein the distributed ledger comprises a record of at least one smart contract associated with the patient behavior record; determine whether the patient behavior record satisfies at least one condition of the smart contract; instruct the transfer of one or more tokens to a patient account associated with the patient, if the at least one condition of the smart contract is satisfied; and update the distributed ledger based on the transfer of one or more tokens, through communication over the blockchain distributed network.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1-27. (canceled)

28. A system for encouraging patient behavior, comprising:
a sensor removably coupleable to a medication container and configured to generate a sensor signal indicating opening or closing of the medication container when the sensor is coupled to the medication container;
an output device configured to provide feedback to a patient in response to the sensor signal; and
a transceiver configured to transmit container data over a network to one or more computing devices, wherein the container data is based at least in part on the sensor signal.

29. The system of claim 28, wherein the output device comprises a display.

30. The system of claim 29, wherein the feedback comprises a visual graphic or message provided on the display.

31. The system of claim 28, wherein the output device comprises an audio device.

32. The system of claim 31, wherein the feedback comprises auditory feedback.

33. The system of claim 32, wherein the auditory feedback comprises an auditory message.

34-35. (canceled)

36. A method for encouraging patient behavior, comprising:
receiving a sensor signal indicating opening or closing of a medication container, the sensor signal generated by a sensor removably coupleable to the medication container;
generating feedback to the patient based on the sensor signal;
providing the feedback to the patient at a mobile device associated with the patient.

37. The method of claim 36, wherein the feedback comprises a displayed graphic or message.

38. The method of claim 36, wherein the feedback comprises auditory information.

39-40. (canceled)

41. The method of claim 36, wherein generating feedback comprises comparing information derived from the sensor signal to a predetermined health regimen.

42. The method of claim 36, further comprising providing a reminder to the patient at the mobile device to engage in an action associated with a predetermined health regimen.

43. A method for encouraging patient behavior, comprising:
receiving medication data generated by a medication monitor of a patient using a computing device comprising a processor and memory;
notifying a set of one or more predetermined contacts based at least on the medication data using the computing device; and
outputting feedback to the patient using the medication monitor based at least in response to the medication data.

44. The method of claim 43, wherein the feedback comprises at least one prompt to modify the patient behavior.

45. The method of claim 44, wherein the prompt may comprise encouragement to comply with a dosage schedule.

46. The method of claim 43, wherein the one or more predetermined contacts comprises one or more of a health care professional, a patient's partner, family member, and support group.

47. The method of claim 43, further comprising establishing a communication channel between the computing device and a health care professional device in response to the medication data.

48. The system of claim 28, wherein the sensor is removably coupleable to a cap of the medication container.

49. The system of claim 48, wherein the sensor comprises at least one of an accelerometer and a gyroscope, and wherein the sensor is configured to generate a sensor signal indicating angular rotation of the cap of the medication container.

50. The system of claim 28, wherein the feedback comprises one or more of a reminder and warning based on a predetermined dosage schedule.

51. The system of claim 28, further comprising a geolocator sensor configured to generate position data of the medication container.
